(19) United States
(12) Patent Application Publication
(10) Pub. No.: US 2020/0163601 A1
(43) Pub. Date: May 28, 2020

(54) OPTOELECTRONIC SENSOR

(71) Applicant: OSRAM OLED GmbH, Regensburg (DE)

(72) Inventor: Mathieu Rayer, Augsburg (DE)

(21) Appl. No.: 16/631,846

(22) PCT Filed: Jul. 18, 2018
(86) PCT No.: PCT/EP2018/069560

§ 371 Date: Jan. 17, 2020

(30) Foreign Application Priority Data
Jul. 19, 2017 (DE) 10 2017 116 308.5

(51) Int. Cl. A61B 5/1455; A61B 5/0205; A61B 5/024; A61B 90/30; A61B 5/0255; A61B 90/00

(52) U.S. Cl. CPC A61B 5/14552; A61B 5/0205; A61B 5/02433; A61B 2090/3614; A61B 2562/0238; A61B 5/0255; A61B 2090/306

(57) ABSTRACT

An optoelectronic sensor may include a radiation source designed to emit electromagnetic radiation, a receiver designed to receive a reflection of the radiation, an optical waveguide optically coupled to the radiation source, and/or to the receiver so that an optical coupling region is formed to couple the radiation out of the sensor and to inject the reflection into the sensor to determine properties of a sample to be determined. The sensor may be configured to rest against a sample.

### CROSS-REFERENCE TO RELATED APPLICATIONS

The present application is a national stage entry according to 35 U.S.C. § 371 of PCT application No.: PCT/EP2018/069560 filed on Jul. 18, 2018; which claims priority to German Patent Application Serial No.: 10 2017 116 308.5, which was filed on Jul. 19, 2017; which is incorporated herein by reference in its entirety and for all purposes.

### TECHNICAL FIELD

An optoelectronic sensor is provided, in particular an optoelectronic sensor for recording a vital function.

### BACKGROUND

It is desirable to provide an optoelectronic sensor which requires little space and allows reliable measurement.

### SUMMARY

According to at least one embodiment, the optoelectronic sensor includes a radiation source. The radiation source is configured to emit electromagnetic radiation. For example, the radiation source is configured to emit electromagnetic radiation which lies in the near-infrared spectral range, at around 940 nm, during operation. For example, the radiation source is adapted to emit a wavelength in the red spectral range, for example around 660 nm. According to further embodiments, the radiation source is for example configured to emit green light radiation, for instance with a wavelength of around 535 nm. The radiation source is, for example, a semiconductor radiation source.

According to at least one embodiment, the sensor includes a receiver. The receiver is configured to receive a reflection of the radiation. For example, precisely one receiver is provided. The receiver is adapted to detect the radiation of the wavelength which is emitted by the radiation source. The receiver may include a plurality of image points or pixels, or be a single-channel receiver. For example, a time division multiplex method is used in order to permit the use of a single-channel receiver. The receiver is, for example, a semiconductor receiver.

According to at least one embodiment, the sensor includes a light waveguide. According to one embodiment, the light waveguide is optically coupled to the radiation source. As an alternative or in addition, according to at least one embodiment, the light waveguide is optically coupled to the receiver. The light waveguide is therefore optically coupled only to the radiation source, only to the receiver or both to the radiation source and to the receiver. The light waveguide includes, for example lightguide fibers or is a two-dimensionally extended body. The light guiding takes place by reflection at the interface because of the different refractive indices of the light waveguide and of the surrounding medium. Light which is coupled into the light waveguide propagates in the light waveguide in particular along the main extent direction of the light waveguide.

According to at least one embodiment, an optical coupling region is formed. In the optical coupling region, radiation may be coupled out from the sensor. For example, the radiation may be coupled out from the light waveguide in the optical coupling region. The reflection may be coupled into the sensor in the optical coupling region, for example directly into the receiver or first into the light waveguide, which then guides the reflection to the receiver. The emission of the radiation from the light waveguide and/or the coupling in of the reflection takes place transversely with respect to the direction in the light is guided in the light waveguide.

By means of the radiation coupled out and the reflection coupled in, according to at least one embodiment it is possible to determine properties of a sample to be examined.

The sensor is, in particular, adapted to bear with the coupling region on the sample to be examined. This means, in particular, that a distance between the light waveguide and/or the radiation source and/or the receiver and the sample to be examined is only small. It is possible for a signal transmission from the radiation source to the receiver to take place only via the sample to be examined. On the optical path which is provided according to the specification and which extends from the radiation source to the receiver, there is for example no free beam length for the radiation. The radiation then travels fully, or for the great majority, for example in a path percentage of at least 90% or 95%, in condensed matter and not in gases or an evacuated region.

The sample to be examined is, in particular, a body part of a living being. For example, the body part is a wrist joint or a finger. In particular, the sample to be examined includes human skin, on which the sensor bears directly thereon in a non-limiting embodiment.

According to at least one embodiment, an optoelectronic sensor includes a radiation source which is configured to emit electromagnetic radiation. A receiver is provided, which is configured to receive a reflection of the radiation. The sensor includes a light waveguide which is coupled to the radiation source and/or to the receiver, so that an optical coupling region is formed in order to couple the radiation out from the sensor and to couple the reflection into the sensor. It is therefore possible to determine properties of a sample to be examined. The sensor is adapted to bear with the coupling region on the sample to be examined.

Using such a sensor, a miniaturized module for determining, for example, pulse frequency and/or oxygen saturation in blood may be produced. Such a sensor is also referred to as a monitor for biological functions or vital functions. In particular by the use of the light waveguide, a more flexible arrangement of the individual elements in the sensor is made possible. This leads in particular, to smaller sizes of the sensor. Furthermore, the accuracy and reliability during measurement may be increased since crosstalk between the radiation source and the receiver can be reduced and the signal-to-noise ratio can thus be improved.

The sensor described here is based, in particular, on the following considerations. Sensors for the determination of vital functions use the fact that radiation is scattered differently by tissue according to how high the oxygen level in the blood is. The reflection of the scattered radiation is measured. If this is carried out over a certain period of time, the heart rate can be measured. Such sensors are used, for example, in wristwatches or armbands. They should therefore have a space requirement that is as small as possible. In conventional sensors, however, the radiation source and the receiver need to have a minimum distance from one another in order to minimize crosstalk and improve the signal-to-noise ratio.

The sensor described here now makes use, inter alia, of the concept that a light waveguide is optically coupled to the radiation source and/or to the receiver. For example, radiation is shined not directly from the radiation source into the sample to be examined, but first into the light waveguide. The radiation is then coupled out from the light waveguide and shined into the sample to be examined. The reflection of the radiation is, for example, received directly by the receiver. As an alternative, it is also possible firstly to couple the reflection into the light waveguide and then to guide it to the receiver by means of the light waveguide.

Using such an optoelectronic sensor, a miniaturized module for determining, in particular, pulse frequency and/or oxygen saturation in blood may be produced. Radiation which is guided in the light waveguide cannot reach the receiver directly. The distance between the radiation source and the receiver may therefore be reduced. In this case, low optical crosstalk is achieved. For a sufficiently good signal-to-noise ratio, the sensor therefore requires only a small installation space. The light waveguide may furthermore outwardly cover the radiation source, the receiver and/or further components of the sensor. The esthetic appearance of the sensor is therefore enhanced.

According to at least one embodiment, the sensor includes a further light waveguide. The light waveguide is configured according to the further light waveguide or has different properties.

According to at least one embodiment, the radiation source and the receiver are each optically coupled to one of the light waveguides. For example, the radiation source is optically coupled to the light waveguide and the receiver is not optically coupled to the light waveguide. The receiver is optically coupled to the further light waveguide and the radiation source is not optically coupled to the further light waveguide. It is therefore possible to arrange the radiation source and the receiver independently of the optical coupling region in the sensor. The light waveguide may be used in order to guide radiation of the light source to the optical coupling region. The further light waveguide may be used in order to guide the reflective radiation from the optical coupling region to the receiver.

According to at least one embodiment, a further radiation source is provided, which is optically coupled to the light waveguide. The further radiation source is, for example, adapted to emit radiation with a different wavelength than the radiation source. In particular, it is therefore possible to use radiation with different wavelengths. For example, the further radiation source is adapted to emit a wavelength in the red spectral range, for example around 660 nm. According to further embodiments, the radiation source is for example configured to emit green light radiation, for instance with a wavelength of around 535 nm.

According to at least one embodiment, the light waveguide includes a structuring in order to scatter radiation. The structuring is, in particular, formed in the optical coupling region so that radiation which is guided by the light waveguide along the main propagation direction of the light waveguide is scattered at the structuring and therefore coupled out from the light waveguide. The structuring is, for example, at least one of printing, laser structuring, embossing and mechanical structuring.

According to at least one embodiment, as an alternative or in addition the sensor includes a scattering element which is in contact with the light waveguide in order to scatter radiation from the light waveguide. In particular, structuring of the light waveguide may then be omitted. The scattering element has, for example, a refractive index which corresponds to the refractive index of the light waveguide. No total reflection therefore takes place in the region in which the light waveguide and the scattering element are in contact. Radiation can leave the light waveguide at these positions.

In particular, the scattering element is arranged in contact with the light waveguide in regions and at a distance from the light waveguide in regions. Radiation therefore emerges only at defined positions from the light waveguide in which the scattering element is in contact with the light waveguide.

According to at least one embodiment, the light waveguide, the radiation source and the receiver are arranged in a plane. The light waveguide is configured in order to guide radiation along the plane. A flat sensor may therefore be produced.

According to at least one embodiment, the light waveguide is configured in such a way that radiation which is oriented transversely with respect to the plane is transmitted through the light waveguide. For example, radiation from the radiation source is initially guided along the plane and then coupled out transversely with respect to the plane. The reflection is likewise oriented transversely with respect to the plane and at least for the most part passes through the light waveguide. The reflection therefore reaches the receiver through the light waveguide without being guided by the light waveguide substantially along the plane.

According to at least one embodiment, the radiation source emits along a plane along which the optical coupling region extends. In particular, the radiation is therefore emitted during operation not directly in the direction of the sample to be examined but along the sample. Only in the coupling region is the radiation deviated and travels in the direction of the sample to be examined.

According to at least one embodiment, the radiation source and/or the receiver are arranged outside the optical coupling region of the sensor. With the aid of the light waveguide, it is possible to establish the coupling region and the position of the radiation source and/or of the receiver independently of one another. For example, both the radiation source and the receiver are arranged outside the optical coupling region of the sensor. According to further embodiments, the radiation source is arranged outside the optical coupling region and the receiver is arranged inside the optical coupling region. According to further embodiments, the receiver is arranged outside the optical coupling region and the radiation source is arranged inside the optical coupling region. At least the component, which is arranged outside the optical coupling region of the sensor is, in particular, coupled to the light waveguide or to the further light waveguide.

According to at least one embodiment, the light waveguide extends over the radiation source and the receiver. It is therefore possible to produce a uniform appearance on the side of the coupling region. The esthetic appearance of the sensor is therefore improved.

According to at least one embodiment, the radiation source includes a semiconductor layer sequence for the radiation generation. The radiation source is an LED or a plurality of LEDs. The radiation source may also include a semiconductor laser. The radiation source may then also include a superluminescent diode (SLED).

According to at least one embodiment, the receiver includes a photodetector. The photodetector is, for example, a photodiode or a CCD (charge-coupled device) sensor. The receiver includes, in particular, at least one semiconductor chip.

### BRIEF DESCRIPTION OF THE DRAWINGS

In the embodiments and figures, components which are the same or of the same type, or which have the same effect, are respectively provided with the same references. The elements represented and their size ratios with respect to one another are not to be regarded as to scale. Rather, individual elements, in particular layer thicknesses, may be represented exaggeratedly large for better understanding.

FIGS. 1 to 5 respectively show a schematic representation of a sensor according to a respective embodiment,

FIGS. 6 to 10 respectively show a schematic representation of a sensor with a radiation path indicated according to a respective embodiment.

### DETAILED DESCRIPTION

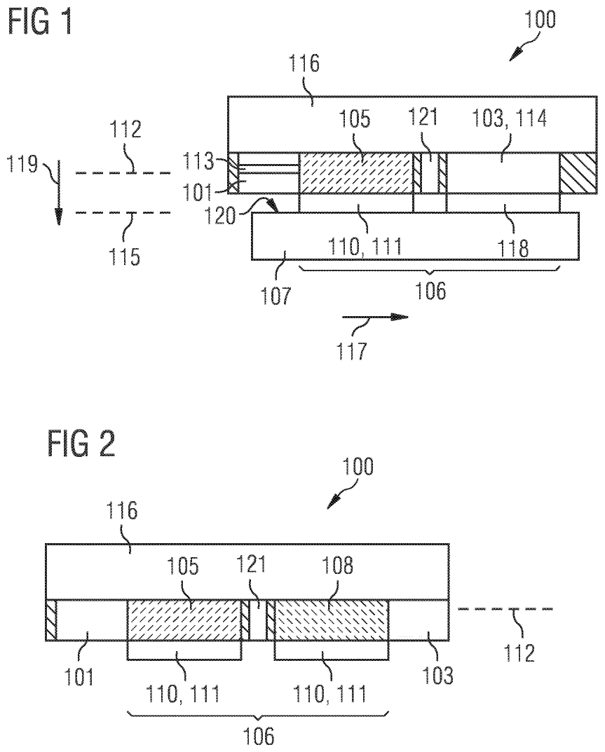

FIG. 1 shows a schematic representation of a sensor 100 according to one embodiment. The sensor 100 is, in particular, configured to record a vital function. For example, the sensor is configured to record a heart rate. As an alternative or in addition, the sensor 100 is configured to record a blood oxygen level. According to further embodiments, as an alternative or in addition, the sensor 100 is configured to determine other properties of a sample 107 to be examined.

The sensor 100 includes a radiation source 101. The radiation source 101 is adapted to generate radiation 102 (FIGS. 6 to 10), in particular electromagnetic radiation in the infrared range to the green range. The radiation source 101 includes in particular an LED, an SLED and/or a semiconductor laser. In particular the radiation source includes a semiconductor layer sequence 113 having at least one active layer for generating the radiation 102.

The sensor 100 includes a receiver 103. The receiver 103 is configured to detect a reflection 104 (FIGS. 6 to 10) of the radiation 102 after the radiation 102 has been reflected or more in the sample 107. The receiver includes, for example, a photodetector.

The radiation source 101 and the receiver 103 are each mechanically coupled to a carrier 116. Electrical coupling is furthermore possible. The carrier 116 is, for example, a circuit board.

The sensor 100 includes a light waveguide 105. The light waveguide is, for example, made of a plastic or a glass. The light waveguide 105 is configured to guide light, i.e. electromagnetic radiation, along its main propagation direction 117. Radiation is totally internally reflected inside the light waveguide at the contact surfaces with the surroundings.

In the embodiment shown, the light waveguide 105 is optically coupled on one side to the radiation source 107. Starting from this side, the light waveguide 105 extends laterally along its main propagation direction 117. The radiation source 101 is also configured to emit radiation laterally, so that the radiation 102 is coupled out from the radiation source 101 into the light waveguide 105. Arranged between the light waveguide 105 and the sample 107 in the vertical direction 119, there is structuring 110, or a scattering element 111. The latter is used for coupling the radiation guided laterally in the light waveguide 105 out in the direction 119 of the sample 107.

Optics 118 are arranged between the receiver 103 and the sample 107. These are used, for example, to deviate the beam path in the direction of the receiver 103.

In the embodiment shown, the radiation source 101, the light waveguide 105 and the receiver 103 are arranged along a common plane 112. The plane 112 corresponds to the horizontal in FIG. 1 and is oriented transversely with respect to the direction 119 and along the direction 117. During operation, the radiation source 101 consequently emits the radiation not in the direction of the sample 107 but along a surface 120 of the sample 107. In the light waveguide 105, the radiation 102 is likewise forwarded along the plane 112 transversely with respect to the direction 119. By means of the structuring 110 and/or the scattering element 111, at least a part of the radiation 102 is deviated in the direction 119 so that the radiation 102 at least partially reaches the sample 107 from the light waveguide 105. The reflection 104 then travels from the sample 107 through the optics 118 to the receiver 103. The receiver 103 is, for example, coupled to an evaluation circuit (not represented) which determines properties of the samples 107 from the signals of the receiver 103.

The sensor 100 is, in particular, in contact with the sample 107 via the structuring 110 and/or the scattering element 111 as well as the optics 118. The sensor 100 bears on the sample 107. The regions, out from which radiation can be coupled, or into which radiation can be coupled, form an optical coupling region 106. The coupling region extends along a plane 115 which is oriented substantially parallel to the surface 120 during operation.

In particular, the optical coupling region 106 is thus defined by the structuring 110 and/or the scattering element 111 as well as the optics 118. Since the radiation 102 is not coupled directly out from the radiation source 101 into the sample 107, it is possible to arrange the radiation source 101 outside the optical coupling region 106.

The sensor 100 includes an optical barrier 121 which, in particular, transmits as little as possible radiation of the wavelength which is emitted by the radiation source 101. Crosstalk between the radiation source 101 and the receiver 103 may therefore be reduced further.

The radiation source 101 is arranged laterally beside the light waveguide 105. The receiver 103 is not optically coupled to the light waveguide 105.

FIG. 2 shows the sensor 100 according to a further embodiment. The sensor 100 of FIG. 2 corresponds substantially to the sensor 100 according to FIG. 1. In contrast to FIG. 1, according to FIG. 2 a further light waveguide 108 is provided. The light waveguide 105 is optically coupled to the radiation source 101. The further light waveguide 108 is optically coupled to the receiver 103.

During operation, the radiation 102 emitted laterally by the radiation source 101 is initially forwarded laterally by the light waveguide 105. The radiation is vertically coupled out at least partially, and is reflected at least partially by the sample 107 (not explicitly represented in FIG. 2). The reflection 104 is initially coupled into the further light waveguide 108. The further light waveguide 108 then guides the reflection 104 to the receiver 103. The receiver 103 is consequently also sensitive in a direction which is oriented transversely with respect to the surface 120 of the sample 107.

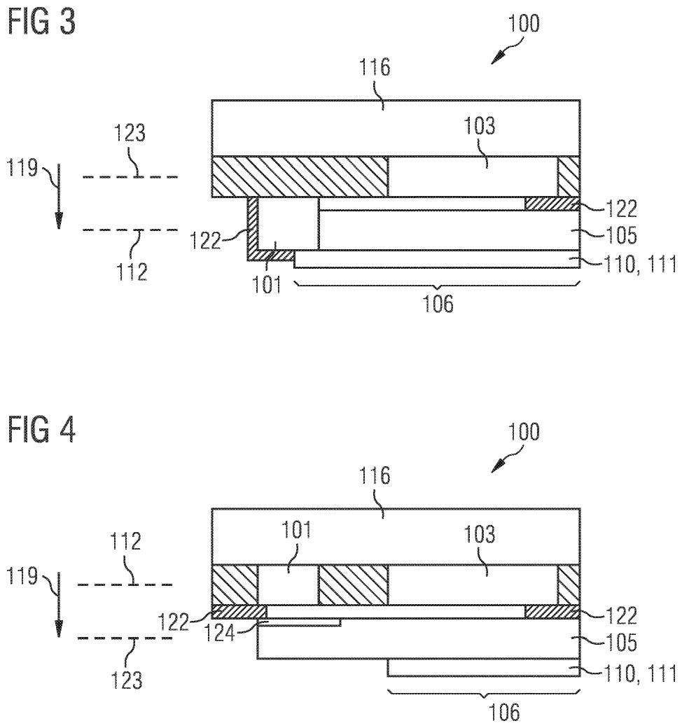

FIG. 3 shows the sensor 100 according to a further embodiment. In contrast to the embodiment of FIG. 1, the receiver 103 is not arranged in the same plane 112 as the light waveguide 105 and the radiation source 101. According to FIG. 3, the receiver 103 is arranged in a further plane 123. The plane 123 is separated from the plane 112 along the direction 119.

The light waveguide extends along the plane 112, starting from the radiation source 101, along the coupling region 106 over the receiver 103. A relatively large coupling region 106 may therefore be produced. A holding element 122 or a plurality of holding elements 122 are provided in order to fasten the light waveguide 105 and the radiation source 101 on the carrier 116.

FIG. 4 shows a further embodiment of the sensor 100. The sensor 100 is constructed in substantially the same way as the sensor 100 according to FIG. 3. In contrast to the sensor of FIG. 3, the sensor according to FIG. 4 includes a radiation source which emits out along the direction 119 transversely with respect to the plane 112. The radiation source 101 and the receiver 103 are arranged in the plane 112. The light waveguide 105 is arranged along the plane 123, which is spaced apart from the plane 112 in the direction 119.

The radiation 102 is emitted in the direction of the sample 107. It does not travel directly to the sample 107, however, but is initially coupled into the light waveguide 105. To this end, the light waveguide includes an input coupling structure 124. The latter may be a structuring of the light waveguide 105 or an extra component which improves the coupling of the radiation 102 from the radiation source 101 into the light waveguide 105.

In the light waveguide, the radiation is then forwarded transversely with respect to the emission direction along the plane 123 in the direction of the coupling region 106. The structuring 110 and/or the scattering element 111 are arranged in the coupling region 106, so that the radiation can leave the light waveguide 105. The receiver 103 is also arranged in the coupling region 106 so that the reflection 104 can travel, starting from the sample 107, through the light waveguide 105 to the receiver 103. The light waveguide 105 extends, in particular, over the radiation source 101 and the receiver 103. In particular, a uniform appearance is therefore produced on that side of the sensor 101 which faces toward the sample during operation.

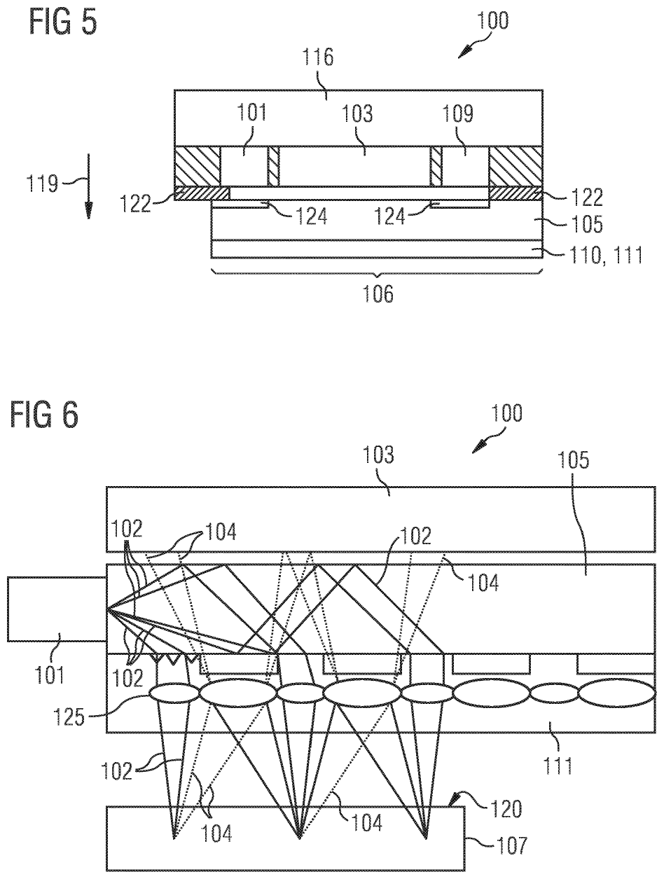

FIG. 5 shows the sensor 100 according to a further embodiment. In addition to the radiation source 101, the sensor 100 according to FIG. 5 includes a further radiation source 109. In particular, the radiation source 101 and the further radiation source 109 are configured to emit radiation with different wavelengths to one another. The radiation source 101 and the further radiation source 109 are each optically coupled to the same light waveguide 105. Radiation of the radiation source 101 is coupled into the light waveguide 105. Radiation of the further radiation source 109 is likewise coupled into the same light waveguide 105. According to further embodiments, two separate light waveguides 105 and 108 are provided, each of which is coupled only to a single radiation source.

The light waveguide 105 extends along the entire optical coupling region 106 and also covers the receiver 103. The receiver is, for example, arranged between the radiation sources 101 and 109. The radiation sources 101 and 109 emit the radiation 102 primarily along the direction 119. The barrier 121 may therefore be omitted. Both the radiation of the radiation source 101 and the radiation of the further radiation source 109 are initially diverted laterally by the light waveguide 105 before they reach the sample 107.

FIG. 6 shows the sensor 100 with the radiation path of the radiation 102 and of the reflection 104 according to one embodiment.

The radiation 102 is initially emitted laterally by the radiation source 101 into the light waveguide 105. There, the radiation 102 is guided, in particular, parallel to the surface 120. In order to couple the radiation out from the light waveguide 105 in the direction of the sample 107, the scattering element 111 is provided. The scattering element 111 includes, in particular, contact regions in which there it is in contact with the light waveguide 105. Between these, the scattering element 111 includes regions in which it is arranged at a distance from the light waveguide 105. In the coupling regions, there is not a sufficiently large refractive index jump, so that the radiation 102 travels from the light waveguide 105 into the scattering element 111 and from there in the direction of the sample 107. In order to influence the beam path further, according to embodiments, the scattering element 111 includes optics 125. The optics 125 are, for example, produced with the aid of materials having different refractive indices. The reflections 104 likewise initially travel again to the scattering element 111 and are forwarded by the scattering element 111 and/or the optics 125 in the direction of the receiver 103.

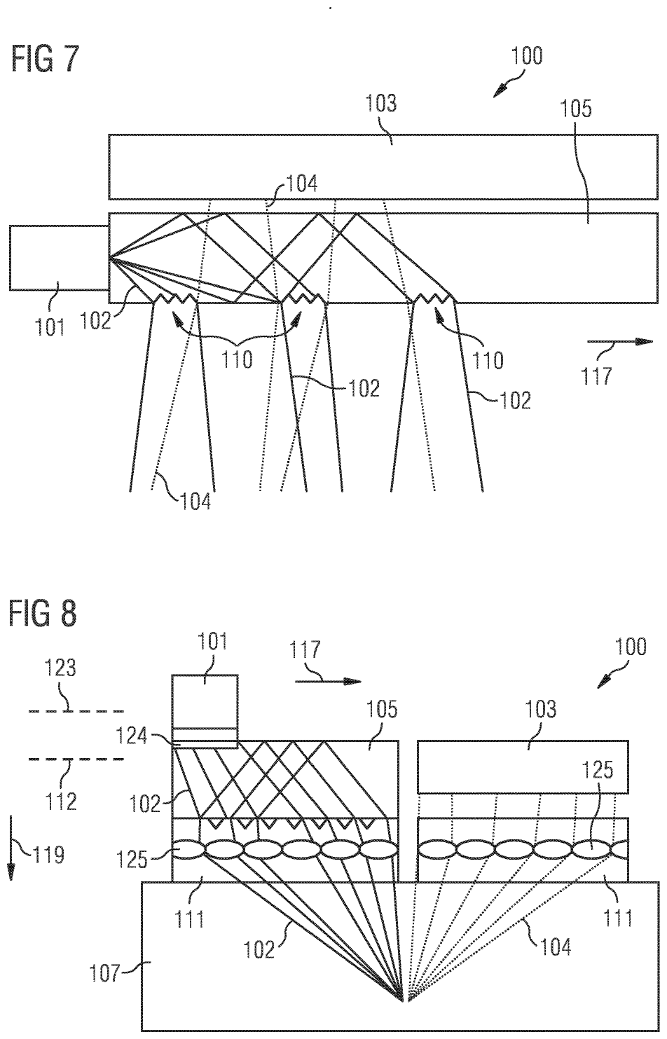

FIG. 7 shows the sensor 100 according to a further embodiment. In contrast to the sensor 100 according to FIG. 6, according to FIG. 7 the separate scattering element 111 is omitted. Instead, the light waveguide 105 itself includes structurings 110. The structurings 110 are, in particular, introduced fully or in regions on that surface of the light waveguide 105 which faces toward the sample 107 during operation. The structurings 110 are used to scatter the radiation 102 transversely with respect to the main propagation direction 117 of the light waveguide 105. The structuring 110 is, for example, a roughening of the surface of the light waveguide 105. The reflection 104 may pass through the light waveguide 105 transversely with respect to its main propagation direction 117, in order to reach the receiver 103.

FIG. 8 shows a further embodiment of the sensor 100. The radiation source 101 is configured to emit the radiation 102 along the direction 119. The radiation 102 is initially coupled into the waveguide 105 and is guided by the latter along its main propagation direction 117. With the aid of the scattering element 111, the radiation 102 is subsequently directed in the direction of the sample 107 again. The reflection 104 is guided to the receiver 103 by means of the further scattering element 111. The two scattering elements 111 are in particular, arranged next to one another along the main propagation direction 117. The light waveguide 105 and the receiver 103 are arranged in a common plane 112. The radiation source 101 is arranged in the spaced-part further plane 123.

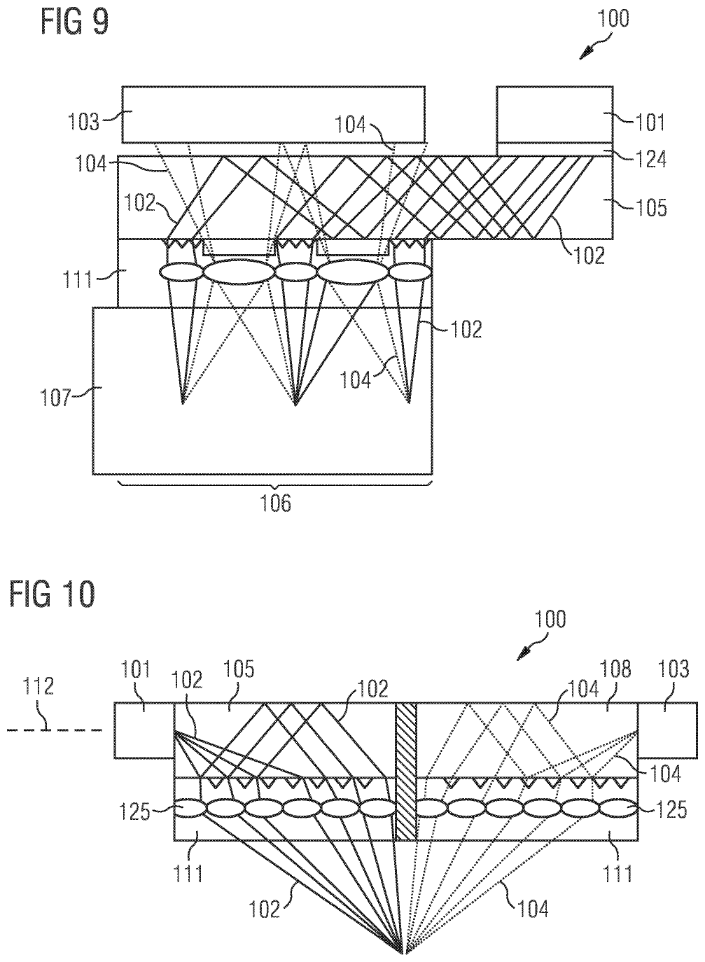

FIG. 9 shows the sensor 100 according to a further embodiment. The radiation source 101 is arranged outside the coupling region 106. The radiation source 106 is arranged in a region of the sensor 100 which, for example, is not in contact with the sample 107. By means of the light waveguide 105, the radiation 102 is guided to the coupling region 106.

FIG. 10 shows the sensor 100 according to a further embodiment. The radiation source 101, the light waveguide 105, the further light waveguide 108 and the receiver 103 are arranged in a common plane 112. The light waveguide 105 is used to guide the radiation 102 along the plane 112. The further light waveguide 108 is used to guide the reflection 104 along the plane 112 to the receiver 103. A respective scattering element 111 having optics 125 is arranged both on the light waveguide 105 and on the further light waveguide 108.

The sensor 100 with the light waveguide 105 allows compact construction with a good signal-to-noise ratio and an improved esthetic appearance. According to one embodiment, the radiation source 101 is optically coupled to the light waveguide 105. According to one embodiment, the light waveguide 105 is arranged on the receiver 103 or next to the receiver 103. According to embodiments, the radiation 102 is coupled out from the light waveguide 105 in the direction of the sample 107 by means of the structuring 110. The structuring 110 includes, in particular, a microstructured profile. The radiation 102 is scattered and reflected in the sample 107, and subsequently collected and guided to the receiver 103. According to embodiments, to this end the light waveguide 105 is either used, i.e. the same optical system. According to further embodiments, the further light waveguide 108 and/or further optics such as the scattering element 111 and the optics 125 are used.

The radiation source 101 may be arranged above the light waveguide 105 (FIGS. 4, 5, 8, 9). According to further embodiments, the radiation source 101 is arranged next to the light waveguide 105 (FIGS. 1, 2, 3, 6, 7, 10). According to embodiments, the input coupling structure 124 is used when the radiation source 101 is arranged above the light waveguide 105, in order to couple the radiation 102 from the light source 101 into the light waveguide 105. The input coupling structure 125 may be configured either to refract light or diffract light.

According to embodiments, a plurality of radiation sources 101, 109 are coupled to the same light waveguide 105. In particular, the radiation sources 101, 109 have different wavelengths to one another of the emitted radiation 102. This is advantageous in particular when measuring the oxygen level of blood. Furthermore, a signal improvement may be achieved.

According to embodiments, the structuring 110 is integrated directly on the light waveguide 105 (FIG. 7). According to further embodiments, as an alternative or in addition, the separate scattering element 111 is provided. According to embodiments, a plurality of optical surfaces are provided on the side facing toward the sample, in order to further improve the signal-to-noise ratio by a predetermined distribution of the radiation intensity and collection of the reflection 104. This is achieved, for example, by a predetermined arrangement of the regions in which the scattering element 111 is in direct contact with the light waveguide 105, and of the regions in which the scattering element 111 is at a distance from the light waveguide 105.

The scattering element 111 and/or the input coupling structure 124 may refract light and/or diffract light.

According to at least one embodiment, the light waveguide 105 includes an antireflection coating or a correspondingly treated surface. This reduces the radiation 102 travelling directly to the receiver 103 instead of into the waveguide 105, for example by Fresnel reflection.

In particular when the sensor 100 is used as a sensor for determining a vital function, when the sensor 100 is in contact with the skin of a human during operation, the contact region of the sensor 100 is configured as flatly as possible without the structuring 110 directly in the light waveguide 105. The functionality of the structure 110 could be impaired by dust and/or grease. The use of the separate scattering element 111 may therefore be advantageous.

The radiation 102 which has been coupled into the light waveguide 105 can no longer travel directly to the receiver 103. Even with a short distance between the receiver 103 and the radiation source 101, crosstalk of the radiation 102 directly to the receiver 103 is therefore sufficiently small. The size of the sensor 101 may therefore be reduced. Furthermore, the light waveguide 105, the scattering element 111 and/or the further optical elements serve as protection for the radiation source 101 and/or the receiver 103. Conventionally additionally provided protective layers may therefore be omitted.

The receiver 103 and/or the radiation source 101, and optionally further components of the sensor 100, are covered in one direction by the light waveguide 105, or the scattering element 111. They are therefore no longer visible, or less visible. The esthetic appearance of the sensor 100 is therefore enhanced. By means of the lightguide structure with the light waveguide 105 and/or the light waveguide 108 and/or the scattering element 111, and optionally further optical layers, it is possible to distribute the distribution of the radiation 102 along the optical coupling region 106 in a predetermined way. Furthermore, recording of the reflections 104 at a fixed angle is possible. An improvement in the signal-to-noise ratio is therefore made possible, and inaccuracies during the measurement are therefore reduced.

The invention is not restricted by the description with the aid of the embodiments to the latter. Rather, the invention covers any new feature and any combination of features, which includes in particular any combination of features in the patent claims, even if this feature or this combination is not explicitly indicated per se in the patent claims or embodiments. In particular, any combination of individual features of the various configurations of the sensor 100 in FIGS. 1 to 10 is possible. The different arrangement and configuration of the radiation source 101, of the receiver 103, of the light waveguides 105 and 108 and of the further radiation source 109 and of the other elements may respectively be combined individually with the configurations of other figures. The various configurations of the elements of the sensor 100 of the various embodiments may be combined with one another in different combinations.

### LIST OF REFERENCES

100 sensor
101 radiation source
102 electromagnetic radiation
103 receiver
104 reflection
105 light waveguide
106 optical coupling region
107 sample
108 further light waveguide
109 further radiation source
110 structuring
111 scattering element
112 plane
113 semiconductor layer sequence
114 photodetector
115 plane of the coupling region
116 carrier
117 main propagation direction
118 optics
119 direction
120 surface
121 barrier
122 holding element
123 plane
124 input coupling structure
125 optics

1. An optoelectronic sensor comprising:
a radiation source configured to emit electromagnetic radiation,
a receiver configured to receive a reflection of the radiation,
a light waveguide optically coupled to the radiation source and/or to the receiver so that an optical coupling region is formed in order to couple the radiation out from the optoelectronic sensor and to couple the reflection into the optoelectronic sensor in order to determine properties of a sample to be examined, wherein the optoelectronic sensor configured to bear with the coupling region on the sample to be examined.

2. The optoelectronic sensor as claimed in claim 1, comprising a further light waveguide so that the radiation source and the receiver are each optically coupled to one of the light waveguides.

3. The optoelectronic sensor as claimed in claim 1, further comprising a further radiation source optically coupled to the light waveguide.

4. The optoelectronic sensor as claimed in claim 1, wherein the light waveguide further comprises a structure in order to scatter the radiation from the light waveguide.

5. The optoelectronic sensor as claimed in claim 1, further comprising a scattering element in contact with the light waveguide at least in regions in order to scatter the radiation from the light waveguide.

6. The optoelectronic sensor as claimed in claim 1, wherein the light waveguide, the radiation source and the receiver are arranged along a plane, and the light waveguide is configured in order to guide the radiation along the plane.

7. The optoelectronic sensor as claimed in claim 6, wherein the light waveguide is configured in such a way that radiation which is oriented transversely with respect to the plane is transmitted through the light waveguide.

8. The optoelectronic sensor as claimed in claim 1, wherein the radiation source emits along a plane along which the optical coupling region extends.

9. The optoelectronic sensor as claimed in claim 1, wherein the radiation source and/or the receiver are arranged outside the optical coupling region of the sensor.

10. The optoelectronic sensor as claimed in claim 1, wherein the light waveguide extends over the radiation source and the receiver.

11. The optoelectronic sensor as claimed in claim 1, wherein the radiation source comprises a semiconductor layer sequence for the radiation generation.

12. The optoelectronic sensor as claimed in claim 1, wherein the receiver comprises a photodetector.

13. The optoelectronic sensor as claimed in claim 1, wherein the optoelectronic sensor is configured to record a vital function.

14. The optoelectronic sensor as claimed in claim 1, wherein the optoelectronic sensor is configured to record a heart rate and/or a blood oxygen level.
